(19) United States
(12) Patent Application Publication
(10) Pub. No.: US 2017/0130267 A1
(43) Pub. Date: May 11, 2017

(54) METHODS FOR PREDICTING ACUTE REJECTION IN HEART RECIPIENTS

(71) Applicants: INSERM (INSTITUT NATIONAL DE LA SANTÉ ET DE LA RECHERCHE MÉDICALE), Paris (FR); UNIVERSITÉ PARIS DIDEROT - PARIS 7, Paris (FR); UNIVERSITÉ PARIS DESCARTES, Paris (FR); ASSISTANCE PUBLIQUE-HÔPITAUX DE PARIS (APHP), Paris (FR)

(72) Inventors: Jean-Paul DUONG VAN HUYEN, Paris (FR); Xavier JOUVEN, Paris (FR); Alexandre LOUPY, Paris (FR); Marion TIBLE, Paris (FR)

(73) Assignees: INSERM (INSTITUT NATIONAL DE LA SANTE ET DE LA RECERRCHE MEDICALE), Paris (FR); UNIVERSITE PARIS DIDEROT - PARIS 7, Paris (FR); UNIVERSITE PARIS DESCARTES, Paris (FR); ASSISTANCE PUBLIQUE-HOPITAUX DE PARIS (APHP), Paris (FR)

(21) Appl. No.: 15/125,355

(22) PCT Filed: Mar. 17, 2015
(86) PCT No.: PCT/EP2015/055553

§ 371 Date: Sep. 12, 2016

(30) Foreign Application Priority Data
Mar. 17, 2014 (EP) 14305373.4

(51) Int. Cl. C12Q 1/68

(52) U.S. Cl. CPC C12Q 1/6883; C12Q 2600/178; C12Q 2600/158; C12Q 2600/118

(57) ABSTRACT

The present invention relates to method for predicting acute rejection in heart recipients. In particular, the present invention relates to a method for predicting acute rejection in a heart recipient comprising the steps consisting of i) determining the expression level (ELi) of at least one miRNAi selected from the group consisting of miR-155, miR-10a, miR-92a and miR-31 in a blood sample obtained from the heart recipient, ii) comparing the expression level (ELi) determined at step i) with a predetermined reference level (ELRi) and iii) and concluding that the recipient has a high risk of developing acute rejection when the level the expression level (ELi) determined at step i) is different (higher or lower) than the predetermined reference level (ELRi).

### FIELD OF THE INVENTION

The present invention relates to method for predicting acute rejection in heart recipients.

### BACKGROUND OF THE INVENTION

Heart transplantation is currently the last option of treatment in end-stage heart failure. Although potent immunosuppressive treatments have reduced the incidence of acute cellular rejection (ACR) in heart recipients, the emergence of antibody mediated rejection (AMR) has redefined the follow-up of heart transplantation recipients. AMR is a pathological process that is associated with pathogenic donor specific anti-HLA antibodies (DSA). Acute AMR can be associated with cardiac dysfunction and high mortality in the absence of specific treatment. Subclinical AMR is also an indolent and chronic process that has been linked to the progression of cardiac allograft vasculopathy (CAV) and bad outcome. Currently, histological assessment of endomyocardial biopsies (EMB) is the gold standard for the diagnosis of acute rejection. Post-transplantation protocols consist of frequent EMBs which sampling procedure is expensive, invasive and is associated with potential risk of complication. In the era of translational medicine the need for non-invasive, sensitive and reliable biomarkers is critical, and may reduce the need for EMB.

Discovery and growing characterization of microRNAs over the last ten years is impacting our ways of understanding gene regulation and signaling pathways. MicroRNAs (miRNAs) are endogenous, single-stranded molecules about 22 nucleotides long which, when associated with their mRNA target in the 3′ UTR region, may repress target genes through degradation of the mRNA and/or inhibition of protein expression. MicroRNAs have been largely studied, especially in cancer, and their deregulation have been shown to be linked to many pathological disorders upon which neoplastic, metabolic and inflammatory conditions. In the heart transplantation field, very few studies have been exploring the role of miRNAs in rejection.

### SUMMARY OF THE INVENTION

The present invention relates to method for predicting acute rejection in heart recipients. In particular, the present invention is defined by the claims.

### DETAILED DESCRIPTION OF THE INVENTION

The inventors studied the expression in endomyocardial biopsies (EMB) from heart recipients of 14 miRNAs chosen from an in silico analysis conjugating relevant bibliography analysis and databases screening. Seven tissue miRNAs could identify rejecting recipients (p<0.001) and some could differentiate ACR from AMR. The inventors also found that the expression of 4 of these miRNAs was regulated in serum concomitant to the EMB. The expression of these 4 circulating miRNA correlated with the expression of tissue miRNA and was able to predict rejection. The present invention therefore depicts a method for prediction acute rejection in heart transplanted recipients.

Accordingly the present invention relates to a method for predicting acute rejection in a heart recipient comprising the steps consisting of i) determining the expression level (EL,) of at least one miRNAi selected from the group consisting of miR-155, miR-10a, miR-92a and miR-31 in a blood sample obtained from the heart recipient, ii) comparing the expression level (EL,) determined at step i) with a predetermined reference level (ELRᵢ) and iii) and concluding that the recipient has a high risk of developing acute rejection when the level the expression level (ELi) determined at step i) is different (higher or lower) than the predetermined reference level (ELRi).

As used herein, the term “heart transplantation” refers to the process of taking a heart, (i.e. “graft”) from one individual and placing it or them into a different individual. The individual who provides the transplant is called the “donor” and the individual who received the transplant is called the “recipient”. Typically, the graft t is an allograft, i.e. a graft, transplanted between two genetically different individuals of the same species.

As used herein, the term “acute rejection” or “AR” is the rejection by the immune system of a tissue transplant recipient when the transplanted tissue is immunologically foreign. It is possible to distinguish antibody mediated rejection (AMR) and acute cellular rejection (ACR). Acute cellular rejection is characterized by infiltration of the transplanted tissue by immune cells of the recipient, which carry out their effector function and destroy the transplanted tissue. AMR is a pathological process that is associated with pathogenic donor specific anti-HLA antibodies (DSA).

By “predicting acute rejection”, it is meant determining the likelihood that the recipient is undergoing, or will rapidly undergo, a rejection response during, or following, the date at which the blood sample was taken. By “a high risk of having acute rejection” it is meant that the recipient has a high probability of developing rapidly, acute rejection during, or following, the date at which the blood sample was taken. In particular, it is meant that the recipient has a probability of at least 85% (i.e. 85, 86, 87, 88, 89, 90, 91, 92, 93, 94, 95, 96, 97, 98, 99 or 100%) of developing acute rejection.

The term “blood sample” means a whole blood, serum, or plasma sample obtained from the recipient. To obtain a blood sample, any technique known in the art may be used, e.g. a syringe or other vacuum suction device. A blood sample can be optionally pre -treated or processed prior to be used. Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolarity regulating reagent, a pH regulating reagent, and/or a cross-linking reagent. Thus, a sample, such as a blood sample, can be analyzed under any of the methods and systems herein within 1 week, 6 days, 5 days, 4 days, 3 days, 2 days, 1 day, 12 hrs, 6 hrs, 3 hrs, 2 hrs, or 1 hr from the time the blood sample is obtained.

Conventional methods and reagents for isolating RNA from a sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction,

TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person.

The term “miRNAs” refers to mature microRNA (non-coding small RNAs) molecules that are generally 21 to 22 nucleotides in length, even though lengths of 19 and up to 23 nucleotides have been reported. miRNAs are each processed from longer precursor RNA molecules (“precursor miRNA”: pri-miRNA and pre-miRNA). Pri-miRNAs are transcribed either from non-protein-encoding genes or embedded into protein-coding genes (within introns or non-coding exons). The “precursor miRNAs” fold into hairpin structures containing imperfectly base-paired stems and are processed in two steps, catalyzed in animals by two

Ribonuclease III-type endonucleases called Drosha and Dicer. The processed miRNAs (also referred to as “mature miRNA”) are assembled into large ribonucleoprotein complexes (RISCs) that can associate them with their target mRNA in order to repress translation. All the miRNAs pertaining to the invention are known per se and sequences of them are publicly available from the data base http://www.mirbase.org/cgi-bin/mirna_summary.pl?org=hsa.

The expression level of one or more miRNA in the blood sample may be determined by any suitable method. Any reliable method for measuring the level or amount of miRNA in a sample may be used. Generally, miRNA can be detected and quantified from the blood sample (including fractions thereof), such as samples of isolated RNA by various methods known for mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g. , hybridization arrays (e.g. , microarrays), NanoString analysis, Northern Blot analysis, branched DNA (bDNA) signal amplification, in situ hybridization, etc.), and sequencing-based methods (e.g. , next- generation sequencing methods, for example, using the Illumina or IonTorrent platforms). Other exemplary techniques include ribonuclease protection assay (RPA) and mass spectroscopy.

In some embodiments, RNA is converted to DNA (cDNA) prior to analysis. cDNA can be generated by reverse transcription of isolated miRNA using conventional techniques. miRNA reverse transcription kits are known and commercially available. Examples of suitable kits include, but are not limited to the mirVana TaqMan® miRNA transcription kit (Ambion, Austin, Tex.), and the TaqMan® miRNA transcription kit (Applied Biosystems, Foster City, Calif.). Universal primers, or specific primers, including miRNA-specific stem-loop primers, are known and commercially available, for example, from Applied Biosystems. In some embodiments, miRNA is amplified prior to measurement. In some embodiments, the expression level of miRNA is measured during the amplification process. In some embodiments, the expression level of miRNA is not amplified prior to measurement. Some exemplary methods suitable for determining the expression level of miRNA in a sample are described in greater hereinafter. These methods are provided by way of illustration only, and it will be apparent to a skilled person that other suitable methods may likewise be used.

Many amplification-based methods exist for detecting the expression level of miRNA nucleic acid sequences, including, but not limited to, PCR, RT-PCR, qPCR, and rolling circle amplification. Other amplification-based techniques include, for example, ligase chain reaction, multiplex ligatable probe amplification, in vitro transcription (IVT), strand displacement amplification, transcription-mediated amplification, RNA (Eberwine) amplification, and other methods that are known to persons skilled in the art. A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a miRNA) may be performed prior to PCR amplification. Reverse transcription reactions include the use of, e.g., a RNA -based DNA polymerase (reverse transcriptase) and a primer. Kits for quantitative real time PCR of miRNA are known, and are commercially available. Examples of suitable kits include, but are not limited to, the TaqMan® miRNA Assay (Applied Biosystems) and the mirVana™ qRT-PCR miRNA detection kit (Ambion). The miRNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence. In some embodiments, custom qRT-PCR assays can be developed for determination of miRNA levels. Custom qRT-PCR assays to measure miRNAs in a sample can be developed using, for example, methods that involve an extended reverse transcription primer and locked nucleic acid modified PCR. Custom miRNA assays can be tested by running the assay on a dilution series of chemically synthesized miRNA corresponding to the target sequence. This permits determination of the limit of detection and linear range of quantitation of each assay. Furthermore, when used as a standard curve, these data permit an estimate of the absolute abundance of miRNAs measured in the samples. Amplification curves may optionally be checked to verify that Ct values are assessed in the linear range of each amplification plot. Typically, the linear range spans several orders of magnitude. For each candidate miRNA assayed, a chemically synthesized version of the miRNA can be obtained and analyzed in a dilution series to determine the limit of sensitivity of the assay, and the linear range of quantitation. Relative expression levels may be determined, for example, according to the 2(-ΔΔ C(T)) Method, as described by Livak et ah, Analysis of relative gene expression data using real-time quantitative PCR and the 2(-ΔΔ C(T)) Method. Methods (2001) December; 25(4):402-8.

In some embodiments, two or more miRNAs are amplified in a single reaction volume. For example, multiplex q-PCR, such as qRT-PCR, enables simultaneous amplification and quantification of at least two miRNAs of interest in one reaction volume by using more than one pair of primers and/or more than one probe. The primer pairs comprise at least one amplification primer that specifically binds each miRNA, and the probes are labeled such that they are distinguishable from one another, thus allowing simultaneous quantification of multiple miRNAs.

Rolling circle amplification is a DNA-polymerase driven reaction that can replicate circularized oligonucleotide probes with either linear or geometric kinetics under isothermal conditions (see, for example, Lizardi et al., Nat. Gen. (1998) 19(3):225-232; Gusev et al, Am. J. Pathol. (2001) 159(1):63-69; Nallur et al, Nucleic Acids Res. (2001) 29(23):E118). In the presence of two primers, one hybridizing to the (+) strand of DNA, and the other hybridizing to the (−) strand, a complex pattern of strand displacement results in the generation of over 10⁹ copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of miRNA sequence and expression level at very low miRNA concentrations (see, for example, Cheng et al., Angew Chem. Int. Ed. Engl. (2009) 48(18):3268-72; Neubacher et al, Chembiochem. (2009) 10(8): 1289-91). miRNA may be detected using hybridization-based methods, including but not limited to hybridization arrays (e.g., microarrays), NanoString analysis, Northern Blot analysis, branched DNA (bDNA) signal amplification, and in situ hybridization.

Microarrays can be used to measure the expression levels of large numbers of miRNAs simultaneously. Microarrays can be fabricated using a variety of technologies, including printing with fine-pointed pins onto glass slides, photolithography using pre-made masks, photolithography using dynamic micromirror devices, inkjet printing, or electrochemistry on microelectrode arrays. Also useful are microfluidic TaqMan Low-Density Arrays, which are based on an array of microfluidic qRT-PCR reactions, as well as related microfluidic qRT-PCR based methods. In one example of microarray detection, various oligonucleotides (e.g., 200+5′-amino- modified-C6 oligos) corresponding to human sense miRNA sequences are spotted on three- dimensional CodeLink slides (GE Health/Amersham Biosciences) at a final concentration of about 20 μM and processed according to manufacturer's recommendations. First strand cDNA synthesized from 20 μg TRIzol-purified total RNA is labeled with biotinylated ddUTP using the Enzo BioArray end labeling kit (Enzo Life Sciences Inc.). Hybridization, staining, and washing can be performed according to a modified Affymetrix Antisense genome array protocol. Axon B-4000 scanner and Gene-Pix Pro 4.0 software or other suitable software can be used to scan images. Non-positive spots after background subtraction, and outliers detected by the ESD procedure, are removed. The resulting signal intensity values are normalized to per-chip median values and then used to obtain geometric means and standard errors for each miRNA. Each miRNA signal can be transformed to log base 2, and a one-sample t test can be conducted. Independent hybridizations for each sample can be performed on chips with each miRNA spotted multiple times to increase the robustness of the data.

Microarrays can be used for the expression profiling of miRNAs. For example, RNA can be extracted from the sample and, optionally, the miRNAs are size-selected from total RNA. Oligonucleotide linkers can be attached to the 5′ and 3′ ends of the miRNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the, capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA. Total RNA containing the miRNA extracted from the sample can also be used directly without size-selection of the miRNAs. For example, the RNA can be 3′ end labeled using T4 RNA ligase and a fiuorophore-labeled short RNA linker. Fluorophore-labeled miRNAs complementary to the corresponding miRNA capture probe sequences on the array hybridize, via base pairing, to the spot at which the capture probes are affixed. The fluorescence intensity of each spot is then evaluated in terms of the number of copies of a particular miRNA, using a number of positive and negative controls and array data normalization methods, which will result in assessment of the level of expression of a particular miRNA. Several types of microarrays can be employed including, but not limited to, spotted oligonucleotide microarrays, pre-fabricated oligonucleotide microarrays or spotted long oligonucleotide arrays.

miRNAs can also be detected without amplification using the nCounter Analysis System (NanoString Technologies, Seattle, Wash.). This technology employs two nucleic acid-based probes that hybridize in solution (e.g., a reporter probe and a capture probe). After hybridization, excess probes are removed, and probe/target complexes are analyzed in accordance with the manufacturer's protocol. nCounter miRNA assay kits are available from NanoString Technologies, which are capable of distinguishing between highly similar miRNAs with great specificity. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Mass spectroscopy can be used to quantify miRNA using RNase mapping. Isolated RNAs can be enzymatically digested with RNA endonucleases (RNases) having high specificity (e.g., RNase T1, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESTMS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside. Matrix-assisted laser desorption/ionization mass spectrometry (MALDI-MS) has also been used as an analytical approach for obtaining information about posttranscriptionally modified nucleosides. MALDI-based approaches can be differentiated from ESTbased approaches by the separation step. In MALDI-MS, the mass spectrometer is used to separate the miRNA. To analyze a limited quantity of intact miRNAs, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR® XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact miRNAs are eluted from the trap column and directly injected into a CI 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for miRNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple miRNAs can be detected in a microarray format using a novel approach that combines a surface enzyme reaction with nanoparticle- amplified SPR imaging (SPRI). The surface reaction of poly(A) polymerase creates poly(A) tails on miRNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for miRNA profiling at attamole levels. miRNAs can also be detected using branched DNA (bDNA) signal amplification (see, for example, Urdea, Nature Biotechnology (1994), 12:926-928). miRNA assays based on bDNA signal amplification are commercially available. One such assay is the QuantiGene® 2.0 miRNA Assay (Affymetrix, Santa Clara, Calif.). Northern

Blot and in situ hybridization may also be used to detect miRNAs. Suitable methods for performing Northern Blot and in situ hybridization are known in the art. Advanced sequencing methods can likewise be used as available. For example, miRNAs can be detected using Illumina ® Next Generation Sequencing (e.g. Sequencing-By-Synthesis or TruSeq methods, using, for example, the HiSeq, HiScan, GenomeAnalyzer, or MiSeq systems

(Illumina, Inc., San Diego, Calif.)). miRNAs can also be detected using Ion Torrent Sequencing (Ion Torrent Systems, Inc., Gulliford, Conn.), or other suitable methods of semiconductor sequencing.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the expression level of the selected miRNA in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected miRNA in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the present invention relates to a method for predicting acute rejection in a heart recipient comprising the steps consisting of i) determining the expression level (EL_{miR155}) of miR-155 in a blood sample obtained from the heart recipient, ii) comparing the expression level (EL_{miR155}) determined at step i) with a predetermined reference level (ELR_{miR155}) and iii) and concluding that the recipient has a high risk of having acute rejection when the expression level (EL_{miR155}) determined at step i) is higher than the predetermined reference level (ELR_{miR155}).

In some embodiments, the present invention relates to a method for predicting acute rejection in a heart recipient comprising the steps consisting of i) determining the expression level (EL_{miR10a}) of miR-10a in a blood sample obtained from the heart recipient, ii) comparing the expression level (EL_{miR10a}) determined at step i) with a predetermined reference level (ELR_{miR10a} ) and iii) and concluding that the recipient has a high risk of having acute rejection when the expression level (EL_{miR100} determined at step i) is lower than the predetermined reference level (ELR_{miR10a}).

In some embodiments, the present invention relates to a method for predicting acute rejection in a heart recipient comprising the steps consisting of i) determining the expression level (EL_{miR92a}) of miR-92a in a blood sample obtained from the heart recipient, ii) comparing the expression level (EL_{miR92a}) determined at step i) with a predetermined reference level (ELR_{miR92a}) and iii) and concluding that the recipient has a high risk of having acute rejection when the expression level (EL_{miR92a}) determined at step i) is higher than the predetermined reference level (ELR_{miR92a}).

In some embodiments, the present invention relates to a method for predicting acute rejection in a heart recipient comprising the steps consisting of i) determining the expression level (EL_{miR31}) of miR-31 in a blood sample obtained from the heart recipient, ii) comparing the expression level (EL_{miR31}) determined at step i) with a predetermined reference level (ELR_{miR31}) and iii) and concluding that the recipient has a high risk of having acute rejection when the expression level (EL_{miR31}) determined at step i) is higher than the predetermined reference level (ELR_{miR31}).

In some embodiments, the present invention relates to a method for predicting acute mediated antibody rejection (AMR) in a heart recipient comprising the steps consisting of i) determining the expression level (EL_{miR155}) of miR-155 in a blood sample obtained from the heart recipient, ii) determining the expression level (EL_{miR10a}) of miR-10a in a blood sample obtained from the heart recipient, ii) comparing the expression levels (EL_{miR155}) and (EL_{miR10a}) respectively determined at step i) and ii) with their respective predetermined reference level (ELR_{miR155}) and (ELR_{miR10a}) and iii) and concluding that the recipient has a high risk of having acute mediated antibody rejection (AMR) when the expression level (EL_{miR155}) determined at step i) is higher than the predetermined reference level (ELR_{miR155}) and when the expression level (EL_{miR10a}) determined at step i) is lower than the predetermined reference level (ELR_{miR10a}).

In some embodiments, the present invention relates to a method for predicting acute cellular rejection (ACR) in a heart recipient comprising the steps consisting of i) determining the expression level (EL_{miR155}) of miR-155 in a blood sample obtained from the heart recipient, ii) determining the expression level (EL_{miR10a}) of miR-10a in a blood sample obtained from the heart recipient, ii) comparing the expression levels (EL_{miR155}) and (EL_{miR10a}) respectively determined at step i) and ii) with their respective predetermined reference level (ELR_{miR155}) and (ELR_{miR10a}) and iii) and concluding that the recipient has a high risk of having acute cellular rejection (ACR) when the expression level (EL_{miR155}) determined at step i) is higher than the predetermined reference level (ELR_{miR155}) and when the expression level (EL_{miR10a}) determined at step i) is higher than the predetermined reference level (ELR_{miR10a}).

The method of the present invention is clinically very useful. The method of the present invention allows the detection of rejection without the help of a biopsy sampling, therefore diminishing the number of invasive acts: if the blood test returns normal, patient without rejection can skip the biopsy (about 70% of patients) and if the blood test shows a deregulation in one of the miRNAs expression, the patient will undergo a biopsy to confirm this diagnosis. The method of the present invention is also suitable for determining whether the recipient is eligible or not to a change in his existing regimen treatment. Typically, this regimen treatment consists of triple therapy regimen comprising a corticosteroid plus a calcineurin inhibitor (e.g. Ciclosporin, Tacrolimus) and an anti-proliferative agent (e.g. Azathioprine, Mycophenolic acid) may be used. mTOR inhibitors (e.g. Sirolimus, Everolimus). In some embodiments, where the subject is predicted to have an acute rejection response, immunosuppressive therapy can be modulated, e.g., increased or altered, as is known in the art for the treatment/prevention of acute rejection. For example, acute rejection may be treated with a short course of high-dose corticosteroids (e.g. Prednisolone, Hydrocortisone), or if this is not enough, a Antibodies against specific components of the immune system can also be added to this regimen, especially for high-risk recipients For example anti-CD40 antibodies may be administered in case of ACR. B cell depleting antibodies may be administered to the recipient in case of AMR. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. AMR can also require blood exchanges (IvIg, plasmatic exchanges) to remove antibodies present in the recipient circulating compartment and targeting the graft. Reciprocally, where the recipient is predicted a low risk of having acute rejection, the immunosuppressive therapy can be reduced in order to diminish the potential for drug toxicity.

The present invention also relates to reagents, systems and kits thereof for practicing one or more of the above-described methods. The subject reagents, systems and kits thereof may vary greatly. Typically, the systems and kits of the invention include probes, primers macroarrays or microarrays as above described. For example, the kit may comprise a set of miRNA probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers (e.g. stem-loop primers) that may be pre-labelled or may contain an affinity purification or attachment moiety. The kit may further comprise amplification reagents and also other suitably packaged reagents and materials needed for the particular amplification protocol. The systems and kits may further include one or more additional reagents employed in the various methods such as, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like. In addition to the above components, the kit of the present invention may further include instructions for practicing the methods of the present invention. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The invention will be further illustrated by the following figures and examples. However, these examples and figures should not be interpreted in any way as limiting the scope of the present invention.

### FIGURES

FIG. 1. Regulation of tissue miRNA expression in heart allograft rejection. QPCR analysis in 60 frozen endomyocardial biopsies: Ctr: control, TCMR: acute cellular rejection, ABMR: antibody-mediated rejection. Analyses were conducted using the fold value (2̂-DDCT method). Statistics were performed by non-parametric tests (Kruskal Wallis and Mann Whitney tests). *0.05 <p<0.01; **0.01<p<0.001; ***0.001<p<0.0001; ****p<0.0001.

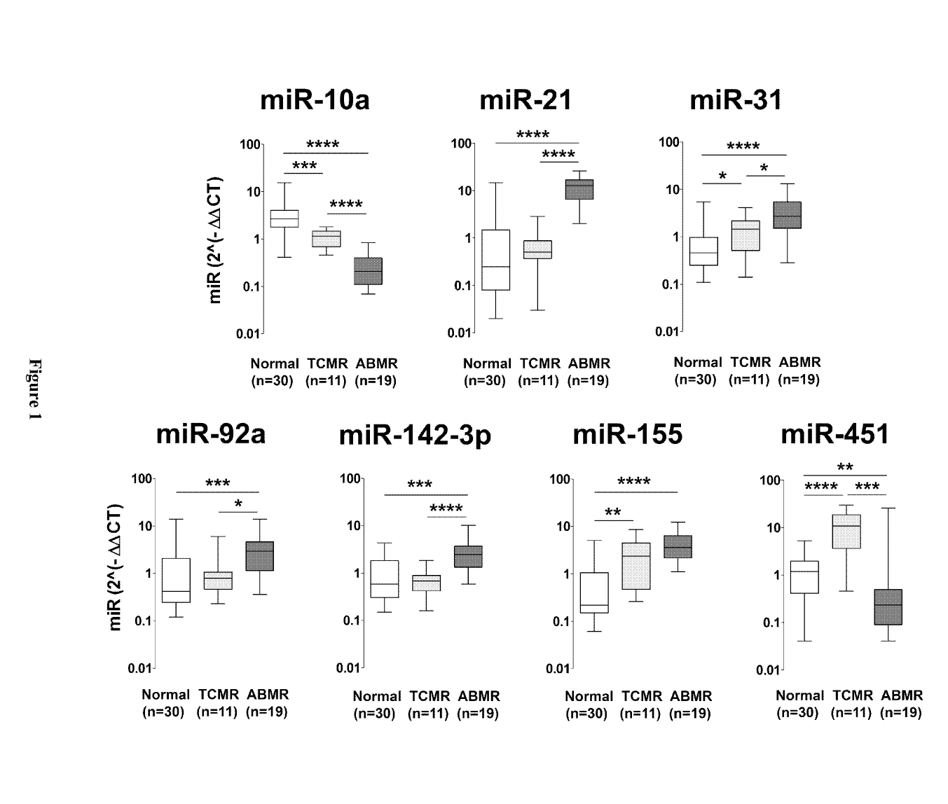

FIG. 2. Correlation between expressions of tissue miRNA and circulating miRNAs. Spearman correlation of miRNAs expression in 28 paired tissue and serum.

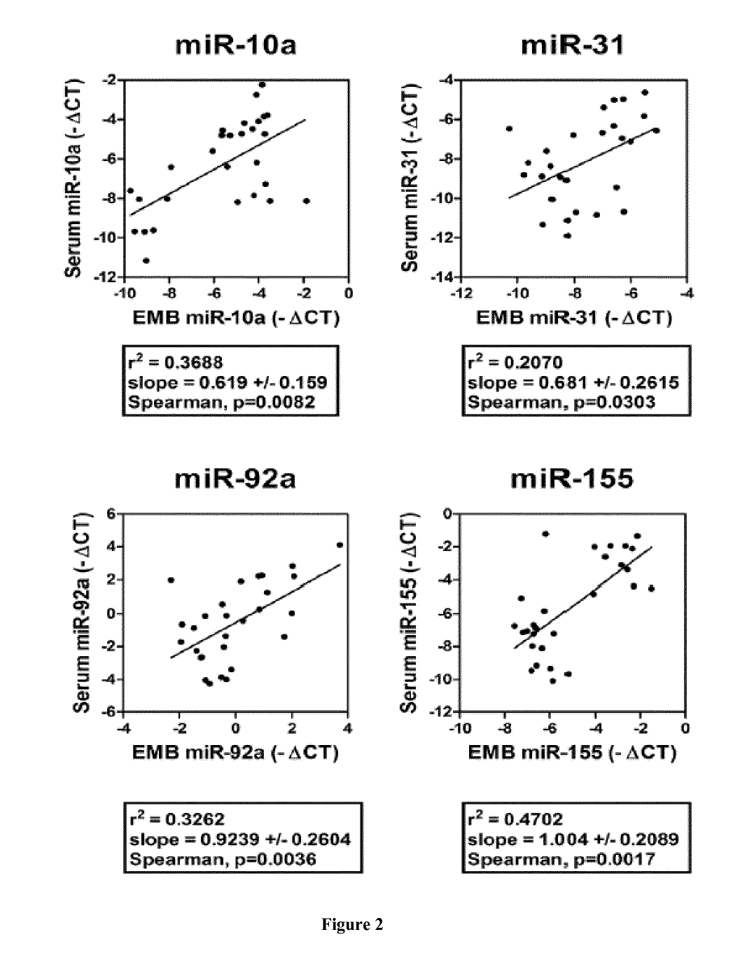

FIG. 3. Levels of circulating miRNA in heart allogra6 rejection. QPCR analysis of the expression of miRNAs in the serum from 60 patients for which concomitant endomyocardial biopsies showed rejection (AR) or were normal (CTR). Analyses were conducted using the fold value. Statistics were performed by non parametric tests. ****p<0.0001. The ROC curve measures the ability of miRNAs expression to classify correctly patients with or without rejection. The accuracy of the test is measured by the area under the curve (AUC).

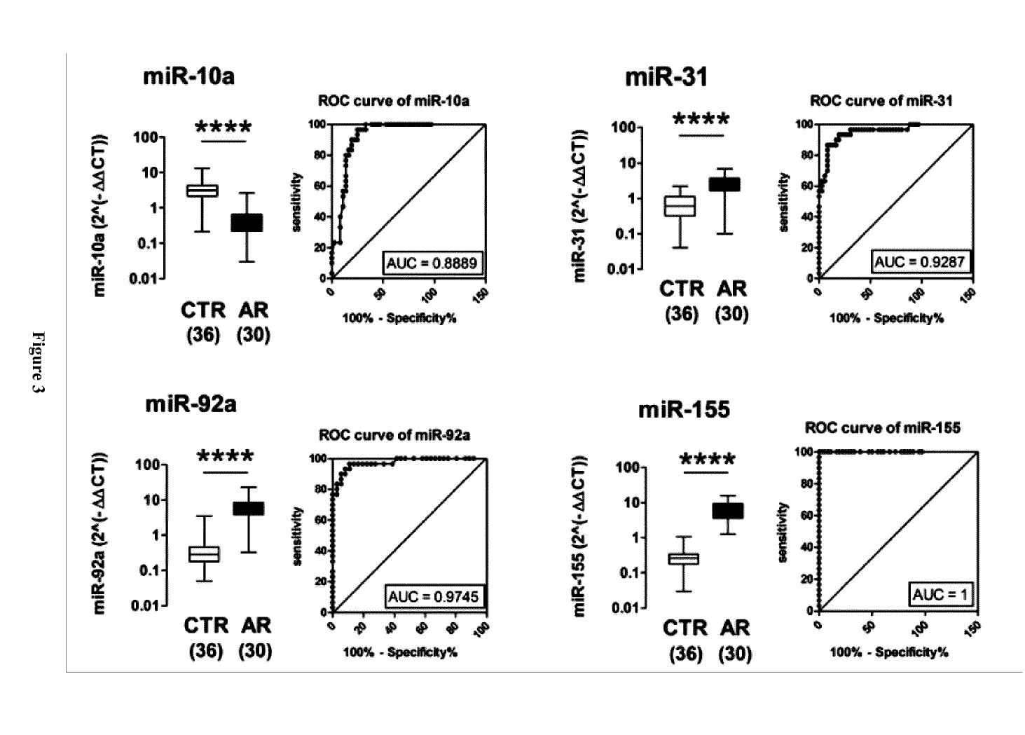

FIG. 4. Scatter plot combining fold values of miR-10a and miR-155 in serum. Association of these two circulating miRNAs was able to differentiate AMR,ACR and controls.

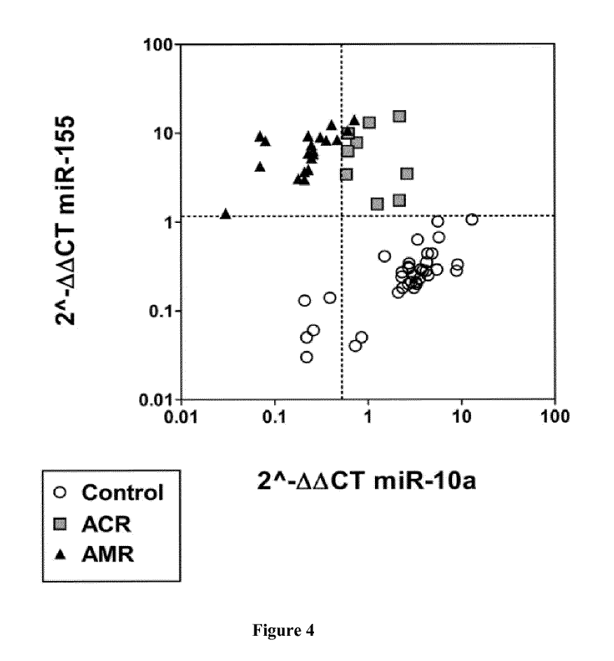

### EXAMPLE

### Methods

This study enrolled 60 heart recipients; A Control group (n=30) included biopsies/serum without rejection; Rejection group (n=30) included 11 biopsies/serum with acute cellular rejection (ACR) and 17 with antibody-mediated rejection (AMR). DSA was also assessed using Luminex single antigen assay.

Fourteen miRNAs were chosen from an in silico analysis conjugating bibliography analysis and databases screening (TargetScan, MiRBase). The expression of these miRNAs was studied by qPCR analysis on frozen EMB and in recipient sera.

### Results

Seven miRNAs showed a significantly different tissue expression during rejection (p<0.001): inflammatory miR-155, 142-3p and 451, endothelial miR-10a and 92a, cardiac miR-21 and 31. All of these 7 miRNAs discriminated AMR from controls (p<0.01) while miR-155, miR-451, miR-10a, and miR-31 differentiated ACR from controls (p=0.03). Three miRNAs (miR-451, miR-10a, and miR-21) were able to discriminate ACR and AMR (p≦0.005).

We then demonstrated significant association (spearman correlation) between the tissue and serum expressions for miR-155 (p<0.0001), miR-10a (p=0.0002), miR-92a (p=0.0317) and miR-31 (p=0.0212).

These four circulating miRNAs were able to discriminate rejecting recipients from controls, solely by the analysis of their expression level in serum. The association of miR-10a and miR-155 was able to distinguish controls, AMR and ACR.

### Conclusion

This study shows the importance of miRNAs as a tool for rejection diagnosis in heart transplantation recipients. The analysis of miRNAs expression level in serum showed the potential role of miRNAs as predictors of rejection and as a significant help for diagnosing rejection without endomyocardial sampling.

### REFERENCES

Throughout this application, various references describe the state of the art to which this invention pertains. The disclosures of these references are hereby incorporated by reference into the present disclosure.

1. A method for predicting acute rejection in a heart transplant recipient comprising the steps of i) determining an expression level (ELi) of at least one miRNAi selected from the group consisting of miR-155, miR-10a, miR-92a and miR-31 in a blood sample obtained from the heart transplant recipient, ii) comparing the expression level (ELi) determined at step i) with a predetermined reference level (ELRi) and iii) and concluding that the heart transplant recipient has a high risk of developing acute rejection when the expression level (ELi) determined at step i) is different than the predetermined reference level (ELRi).

2. The method of claim 1 wherein the at least one miRNAi is miR-155 and wherein the heart transplant recipient has a high risk of having acute rejection when an expression level for miR-155 (EL_{miR155}) determined at step i) is higher than a predetermined reference level for miR-155 (ELR_{miR155}).

3. The method of claim 1 wherein the at least one miRNAi is miR-10a and wherein the heart transplant recipient has a high risk of having acute rejection when an expression level for miR-10a (EL_{miR10a}) determined at step i) is lower than a predetermined reference level for miR10a (ELR_{miR10a}).

4. The method of claim 1 wherein the at least one miRNAi is miR-92a and wherein the heart transplant recipient has a high risk of having acute rejection when an expression level for miR-92a (EL_{miR92a}) determined at step i) is higher than a predetermined reference level for miR-92a (ELR_{miR92a}).

5. The method of claim 1 wherein the at least one miRNAi is miR-31 and wherein the heart transplant recipient has a high risk of having acute rejection when an expression level for miR-31 (EL_{miR31}) determined at step i) is higher than a predetermined reference level for miR-31 (ELR_{miR31}).

6. The method of claim 1 wherein the at least one miRNAi includes miR-155 and miR-10a and wherein the heart transplant recipient has a high risk of having acute mediated antibody rejection (AMR) when an expression level for miR-155 (EL_{miR55}) determined at step i) is higher than a predetermined reference level for miR-155 (ELR_{miR155}) and an expression level for miR-10a (EL_{miR10a}) determined at step i) is lower than the predetermined reference level for miR-10a (ELR_{miR10a}).

7. The method of claim 1 wherein the at least one miRNAi includes miR-155 and miR-10a and wherein the heart transplant recipient has a high risk of having acute cellular rejection (ACR) when the expression level for miR-155 (EL_{miR155}) determined at step i) is higher than a predetermined reference level for miR-155 (ELR_{miR55}) and an expression level for miR-10a (EL_{miR10a}) determined at step i) is higher than a predetermined reference level for miR-10a (ELR_{miR10a}).

8. A method for determining whether a heart transplant recipient is eligible for treatment of acute rejection comprising
i) predicting acute rejection by
a) determining an expression level (ELi) of at least one miRNAi selected from the group consisting of miR-155, miR-10a, miR-92a and miR-31 in a blood sample obtained from the heart transplant recipient, b) comparing the expression level (ELi) determined at step i) with a predetermined reference level (ELRi) and c) concluding that the heart transplant recipient has a high risk of developing acute rejection when at least one expression level (ELi) determined at step a) is different than the predetermined reference level (ELRi); and
ii) determining that the heart transplant recipient is eligible for said treatment when it is concluded that the heart transplant recipient has a high risk of developing acute rejection.

9. The method of claim 8 wherein the treatment comprises administering a short course of high-dose corticosteroids.

10. The method of claim 8 wherein the treatment comprises administering at least one antibody selected from the group consisting of anti-CD40 antibodies, anti-CD20 monoclonal antibodies, anti-CD22 antibodies, anti-CD27 antibodies, anti-CD 19 antibodies anti-APRIL antibodies and anti-IL-6 antibodies.

11. The method of claim 8 wherein the treatment requires includes blood exchanges to remove antibodies targeting the graft which are present in a circulating compartment of the heart transplant recipient.

12. The method of claim 9, wherein the corticosteroids include Prednisolone and/or Hydrocortisone.

13. A method for treating acute rejection in a heart transplant recipient comprising
i) predicting acute rejection by
a) determining an expression level (ELi) of at least one miRNAi selected from the group consisting of miR-155, miR-10a, miR-92a and miR-31 in a blood sample obtained from the heart transplant recipient,
b) comparing the expression level (ELi) determined at step i) with a predetermined reference level (ELRi) and
c) concluding that the heart transplant recipient has a high risk of developing acute rejection when at least one expression level (ELi) determined at step a) is different than the predetermined reference level (ELRi); and, when it is concluded that the heart transplant recipient has a high risk of developing acute rejection,
ii) treating the heart transplant recipient for acute rejection.

14. The method of claim 13 wherein the step of treating comprises one or more of:
administering a short course of high-dose corticosteroids;
administering at least one antibody selected from the group consisting of anti-CD40 antibodies, anti-CD20 monoclonal antibodies, anti-CD22 antibodies, anti-CD27 antibodies, anti-CD19 antibodies anti-APRIL antibodies and anti-IL-6 antibodies; and
blood exchanges to remove antibodies targeting the graft which are present in a circulating compartment of the heart transplant recipient.

15. A method for determining an expression level (ELi) of at least one miRNAi selected from the group consisting of miR-155, miR-10a, miR-92a and miR-31 in a blood sample obtained from a heart transplant recipient, comprising
measuring a level of the at least one miRNA using: an amplification-based method, a hybridization-based method, a sequencing-based method, a ribonuclease protection assay (RPA) or mass spectroscopy.

16. The method of claim 15, wherein the amplification-based method is quantitative polymerase chain reaction (qPCR).

17. An analysis method, comprising the steps of:
obtaining a blood sample from a subject identified as being a potential heart transplant recipient or that has received a heart transplant; and
determining an expression level (Eli) of at least one miRNAi selected from the group consisting of miR-155, miR-10a, miR-92a, and miR-31 in the blood sample.
